(19) United States
(12) Patent Application Publication
(10) Pub. No.: US 2026/0000586 A1
(43) Pub. Date: Jan. 1, 2026

(54) PREVENTING DENTAL CARIES, IMPROVING THE MECHANICAL STRENGTH OF TEETH; AND REDUCING ORAL SENSITIVITY

(71) Applicants: Roger L. Gindlesperger, Layton, UT (US); Mary Ann B. Gindlesperger, Layton, UT (US)

(72) Inventors: Roger L. Gindlesperger, Layton, UT (US); Mary Ann B. Gindlesperger, Layton, UT (US)

(21) Appl. No.: 19/321,526

(22) Filed: Sep. 8, 2025

Related U.S. Application Data
(62) Division of application No. 18/216,036, filed on Jun. 29, 2023.
(60) Provisional application No. 63/357,348, filed on Jun. 30, 2022.

(51) Int. Cl. A61K 8/24; A61K 8/04; A61K 8/34; A61K 8/49; A61Q 11/00

(52) U.S. Cl. CPC A61K 8/24; A61K 8/042; A61K 8/345; A61K 8/4953; A61Q 11/00

(57) ABSTRACT

The present formulation provides a toothpaste or dentifrice that contains Nano-Hydroxyapatite, Micro-Hydroxyapatite, and Theobromine (with or without Xylitol) to provide a multifaceted approach to prevent caries development, promote caries remineralization (improving the mechanical strength of teeth), and treat tooth hypersensitivity.

This application is a divisional of, and claims priority to, U.S. patent application Ser. No. 18/216,036, filed on Jun. 29, 2023, and to U.S. Provisional Patent Application Ser. No. 63/357,348 filed on Jun. 30, 2022, both of which are incorporated by reference herein in their entirety.

### BACKGROUND

Dental decay is a common dental problem that results from the demineralization of the tooth structure due to the acid produced by bacteria in the mouth. This condition can lead to the loss of the protective enamel layer of the teeth, exposing the underlying dentin layer. When the dentin layer is exposed, the teeth become hypersensitive to various stimuli such as heat, cold, acidic substances, and sweets. This dental hypersensitivity can cause significant discomfort and pain to the individual.

While there are numerous treatments available to treat dental hypersensitivity, including desensitizing toothpaste, dental fillings, and fluoride treatments, the reality is that fluoride is the only natural solution that prevents dental decay and aids in the remineralization of the tooth structure. For many decades, the public has been offered the choice of two kinds of toothpaste. One toothpaste includes fluoride and the other is fluoride-free. Fluoride was first introduced into drinking water in the mid 1940s after a study showed improved oral health outcomes, especially improving the strength of tooth enamel. Fluoride works by increasing the resistance of the tooth enamel to acid attacks from bacteria in the mouth. It also helps to incorporate calcium and phosphate into the tooth structure, strengthening it.

However, the use of fluoride has been a subject of controversy in recent times, with some individuals arguing that it may have adverse health effects when consumed in excessive amounts. This has led to an increased demand for natural alternatives to fluoride that can prevent dental decay and alleviate dental hypersensitivity.

Despite widespread scientific endorsement, promotion by the American Dental Association, and substantially all dentists, some people believe that the presence of fluoride in toothpaste is risky, and so prefer to have fluoride-free toothpaste. However, most experts agree that fluoride-free toothpaste does nothing to fight cavities or strengthen enamel, so effectively the use of fluoride-free toothpaste is little better than brushing with just water.

Some toothpaste brands have replaced fluoride with Hydroxyapatite. This is a form of Calcium Apatite that is the same compound that makes up over 95% of tooth enamel, and a large percentage of the dentin of human teeth. Hydroxyapatite in toothpaste is typically in nano-sized particles which are absorbed by the teeth and fill enamel fissures caused by demineralization. Thus, Hydroxyapatite remineralizes a tooth from within, often to the deepest parts of a cavity, binding with plaque and protecting the teeth from harmful bacteria.

Another prior art approach to a fluoride substitute is the use of Theobromine (3,7-dimethylxanthine), surprisingly, a compound found in chocolate. Studies have shown that Theobromine causes formation of large crystallites. It is believed that Theobromine is effective in a toothpaste and is safe even if swallowed accidentally.

Thus, there is a need for a fluoride-free toothpaste that still provides the benefits of fluoride toothpaste.

### SUMMARY

The present formulation provides a toothpaste or dentifrice that contains the combination of Hydroxyapatite and Theobromine (with or without Xylitol) to provide a multifaceted approach to prevent and counteract demineralization of teeth.

One embodiment describes a method of enhancing the indentation hardness of at least one tooth of a mammal in need thereof, said method comprising: a dentifrice formulation comprising: Hydroxyapatite, and Theobromine, topically administer said composition to at least one tooth of said mammal, wherein said composition is selected from the group consisting of a toothpaste, mouthwash, oral paste, and oral gel.

### DETAILED DESCRIPTION OF A PREFERRED EMBODIMENT

Dental caries is one of the most common dental issues that affect people worldwide. The use of materials, such as fluoride, to curb the spread of dental caries has been quite effective. However, recent studies suggest that natural ingredients like Nano-hydroxyapatite, theobromine, and xylitol may be efficient in preventing dental caries, strengthening teeth, and reducing oral sensitivity.

Among the three natural products mentioned, Nano-hydroxyapatite has been found to be very effective in preventing dental caries. Nano-hydroxyapatite is a calcium-phosphorous compound that makes up our teeth enamel. According to research, Nano-hydroxyapatite has remineralization capabilities, which means that it can reverse the early stages of dental caries by restoring the natural minerals in teeth. Nano-hydroxyapatite creates a protective layer that prevents bacterial growth and strengthens weakened teeth to prevent tooth decay. Moreover, it effectively treats damaged enamel to prevent the development of dental cavities, thus reducing the need for dental fillings and tooth extraction.

Theobromine is another natural product that has been found to protect teeth from decay. Theobromine, like caffeine, is a naturally occurring alkaloid found in cocoa beans. Studies have shown that theobromine can help to harden teeth's enamel, making it stronger and less prone to decay. This natural ingredient works by increasing saliva levels, which are essential in neutralizing the harmful acids that cause tooth decay. This, in turn, inhibits the growth and reproduction of bacteria in the oral cavity and breaks down plaque formation on the teeth.

Xylitol is another potent natural ingredient that helps to prevent dental caries, strengthen teeth and reduce oral sensitivity. Xylitol is a natural sugar-alcohol that suppresses the growth of bacteria in the mouth, thereby reducing the likelihood of tooth decay. Research has shown that by reducing the amount of harmful bacteria in one's mouth, xylitol lowers the risk of gum disease, which is a leading cause of tooth loss in adults. Additionally, regular use of xylitol strengthens teeth by increasing mineral density in the teeth, resulting in stronger and healthier teeth.

Collectively, the combination of Nano-Hydroxyapatite, theobromine, and xylitol provides an effective solution for combating dental caries, strengthening teeth, and reducing oral sensitivity. While Nano-Hydroxyapatite prevents dental caries by rebuilding and strengthening teeth enamel, theobromine helps to harden teeth's enamel, inhibits harmful bacteria growth, and suppresses plaque formation. On the other hand, xylitol suppresses the growth of harmful bacteria and increases mineral density in teeth. This natural combination is an effective and safer alternative to the synthetic materials used to protect teeth and prevent common dental issues.

Dental health is essential for overall well-being. The combination of Nano-Hydroxyapatite, theobromine, and xylitol offers a holistic approach to protecting teeth from decay, strengthening teeth, and reducing oral sensitivity.

A dentifrice is described that has a formulation including Nano-Hydroxyapatite, Micro-Hydroxyapatite, and Theobromine (with or without Xylitol) to provide a multifaceted approach to prevent and counteract demineralization of teeth.

Hydroxyapatite makes up 97% of our enamel. Micro-Hydroxyapatite and Nano-Hydroxyapatite are considered to be safe, non-toxic, and very effective for strengthening enamel. This is paired with Theobromine, an alkaloid present in chocolate or Theobroma cacao, which can form Hydroxyapatite crystallites of an increased size that strengthen the enamel, making it less susceptible to acid attack. Acid attacks can eventually lead to cavitation. By increasing the size of apatite crystals that form and strengthen enamel, Theobromine makes teeth less vulnerable to bacterial acid erosion that can eventually lead to cavities. By combining the two ingredients, the compound provides a more proactive approach to remineralization by delivering Hydroxyapatite (Micro-Hydroxyapatite/Nano-Hydroxyapatite) and forming Hydroxyapatite crystallites (Theobromine), thereby doubling down on the efficacy in two proven ways; thereby creating a product that will strengthen teeth on par with, or better than fluoride.

Studies have shown that 15% of Nano-Hydroxyapatite can be as effective at remineralization of initial caries lesions as a highly concentrated fluoride gel. However, health agencies, such as the 2023 European Commission Scientific Committee on Consumer Safety (SCCS) consider Nano-Hydroxyapatite safe at concentrations up to 10% and composed of rod-shaped particles (as opposed to needle shaped particles, which should not be used). Thus, there is a need to provide an effective dentifrice without exceeding recommended percentages of Nano-Hydroxyapatite.

In one embodiment, the formulation combines the maximum recommended percentage of Nano-Hydroxyapatite (10%) with Micro-hydroxyapatite and theobromine to provide improved efficacy above and beyond a dentifrice that contains only 10% Nano-Hydroxyapatite.

In one embodiment the formulation contains Nano-Hydroxyapatite in at least 1%, in at least 2%, in at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20% by weight of the formulation.

In one embodiment the formulation contains Micro-Hydroxyapatite in at least 1%, at least 2%, at least 3%, at least 4%, at least 5%, at least 6%, at least 7%, at least 8%, at least 9%, at least 10%, by weight of the formulation.

In one embodiment, the formulation contains Theobromine in at least 0.1%, at least 0.2%, at least 0.3%, at least 0.4%, at least 0.5%, at least 0.6%, at least 0.7%, at least 0.8%, at least 0.9%, at least 1.0 by weight of the formulation.

In one embodiment, the formulation contains Xylitol in at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25% by weight of the formulation.

In use, Nano-Hydroxyapatite and Micro-Hydroxyapatite both release Calcium and Phosphate ions. These ions assist the efficacy of the Theobromine, which, in the presence of Calcium and phosphate, forms Hydroxyapatite crystallites of an increased size. Those crystallites strengthen the enamel of a tooth, making it less susceptible to acid attack. Xylitol can induce remineralization of deeper layers of demineralized enamel by facilitating Calcium ion movement and accessibility (the ability of the tooth to absorb the hydroxyapatite).

In one embodiment, the compound is comprised of Glycerin, Sorbitol, Water (Aqua), Hydrated Silica, Xylitol, Calcium Carbonate, Flavor (Coconut+Vanilla Mint), Cocamidopropyl Betaine, Micro-Hydroxyapatite, Theobromine, Cocos Nucifera (Coconut) Oil, Aloe Barbadensis Leaf Extract, Panax Ginseng Root Extract, Melaleuca Alternifolia (Tea Tree) Leaf Oil, Stevia Rebaudiana Leaf/Stem Extract, Mentha Piperita (Peppermint) Oil, Camellia Sinensis Leaf Extract, Menthol, Mentha Arvensis Leaf Oil, Xanthan Gum, Potassium Sorbate, Sodium Bicarbonate, Sodium Cocoyl Glutamate, Citric Acid, Charcoal Powder, Titanium Dioxide, and Mica.

In one embodiment the compound is comprised of Glycerin, Sorbitol, Sident 22S, Water (Aqua), Xylitol, Calcium Carbonate, Sident9, Flavor (Coconut, e.g. Bell 41.22377), Cocamidopropyl Betaine, Stevia, Sodium Cocyoyl Glutamate, Timica, Peppermint Oil India (Mentha Piperita), Xanthan Gum, Vanilla Mint Flavor (Frutarom 3220319991.00) Charcoal, Coconut Oil, Potassium Sorbate, Sodium Bicarbonate, Menthol, Peppermint (Cornmint) Oil (Mentha Arvensis), Citric Acid, Micro-Hydroxyapatite, Theobromine, Black Tea Extract, Aloe, Panax Root Ginseng, Green Tea Extract, and Tea Tree Oil.

In one embodiment the compound is comprised of Glycerin, Sorbitol, Sident 22S, Water (Aqua), Xylitol, Calcium Carbonate, Sident9, Flavor (Coconut, e.g. Bell 41.22377), Cocamidopropyl Betaine, Stevia, Sodium Cocyoyl Glutamate, Peppermint Oil India (Mentha Piperita), Xanthan Gum, Vanilla Mint Flavor (Frutarom 3220319991.00), Coconut Oil, Potassium Sorbate, Sodium Bicarbonate, Menthol, Peppermint (Cornmint) Oil (Metntha Arvensis), Citric Acid, Micro-Hydroxyapatite, Theobromine, Black Tea Extract, Aloe, Panax Root Ginseng, Green Tea Extract, Tea Tree Oil, and Nano-Hydroxyapaptite.

In another embodiment, the compound may further include one or more of Coral Calcium, Nano Silver, Arginine Bicarbonate, Gold, Chondrus Crispus (Seaweed), Cranberry Seed Oil, Curcuma Longa (White Turmeric) Root Extract, Vitamin C, Vitamin E, and CoQ10.

In one embodiment, the present disclosure provides a composition, the composition comprising micro-hydroxyapatite, theobromine, xylitol, and a pH of about 6.0 to about 8.0.

In one embodiment, the present disclosure provides a composition, the composition comprising Nano-hydroxyapatite, micro-hydroxyapatite, theobromine, xylitol, and a pH of about 6.0 to about 8.0.

Thus, an improved dentifrice has been described.

What is claimed is:

1. A dentifrice formulation comprising Hydroxyapatite and Theobromine.

2. The dentifrice formulation of claim 1 further including Xylitol.

3. The dentifrice formulation of claim 2 wherein the Hydroxyapatite comprises Nano-Hydroxyapatite and Micro-Hydroxyapatite.

4. The dentifrice formulation of claim 3 wherein the Nano-Hydroxyapatite comprises 3% to 20% by weight of the formulation.

5. The dentifrice formulation of claim 4 wherein the Micro-Hydroxyapatite comprises 1.0% to 10.0% by weight of the formulation.

6. The dentifrice formulation of claim 5 wherein the Theobromine comprises 0.1% to 1.0% by weight of the formulation.

7. The dentifrice formulation of claim 6 wherein the Xylitol comprises 10% to 25% by weight of the formulation.
